(19) United States
(12) Patent Application Publication
(10) Pub. No.: US 2012/0010315 A1
(43) Pub. Date: Jan. 12, 2012

(54) HIGH REFRACTIVE ACRYLATE AND THE METHOD FOR PREPARING THE SAME

(75) Inventors: Hak-June RHEE, Yongin-si (KR); Jeong-Eun BAE, Anyang-si (KR)

(73) Assignee: IUCF-HYU (Industry-University Cooperation Foundation Hanyang University), Seoul (KR)

(21) Appl. No.: 12/833,150

(22) Filed: Jul. 9, 2010

(51) Int. Cl. C08F 2/46; C07C 67/40; C08F 220/26; C07C 69/54

(52) U.S. Cl. 522/33; 560/221; 560/208; 522/181; 522/167; 522/168; 526/320

(57) ABSTRACT

Provided is an acrylate having a high refractive index, which is represented by Chemical Formula (1) or (2), and a method for preparing the same. Since the acrylate has a refractive index, it may be widely applicable to components of display devices such as prism sheet and may be prepared simply, effectively and economically.

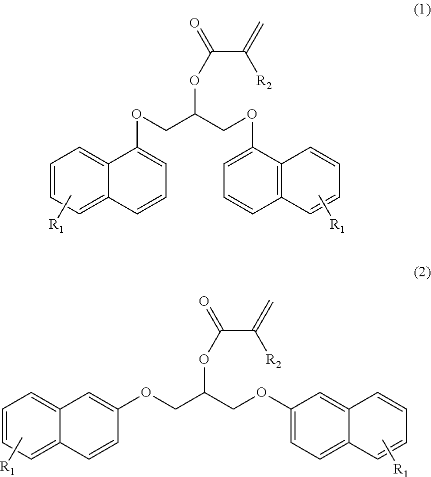

### TECHNICAL FIELD

The present invention relates to an acrylate and a method for preparing the same, more particularly to a novel acrylate having a high refractive index that can be used for display devices such as an optical film, a method for preparing the same, a photocurable resin composition including the acrylate monomer, and an optical film using the same.

### BACKGROUND

High refractive organic materials are widely used for display devices, including a prism sheet or a light guide plate of a liquid crystal display (LCD), a film material for an organic light-emitting diode (OLED), an AR film material of a plasma display panel (PDP), an optical lens, or the like.

Among them, the prism sheet is used to improve brightness of a backlight unit provided on the backside of the LCD. In order to effectively improve the brightness of the backlight unit, flow of light needs to be controlled adequately. The flow of light can be controlled by employing a geometric structure capable of adequately modifying scattering, diffraction, polarization and photon characteristics of light. It is also known that, through variation of physical properties of the material constituting the geometric structure, the flow of light can be further controlled so as to align photons along a desired direction and thus improve brightness along that direction.

Another important optical parameter of the material constituting the prism layer of the prism sheet is refractive index. Since the performance of the prism film is improved as the refractive index is higher, a prism sheet having a high refractive index may be used to improve the efficiency of the LCD backlight.

In general, free radical-polymerizable polymer resins, particularly photocurable resins, are frequently used for the prism layer of the prism sheet. Typical examples of photocurable polymer materials having a high refractive index include (meth)acrylates having one or two aromatic group(s) and (meth)acrylates containing halogen or sulfur. Such high refractive polymer resins are used to prepare prism sheets for backlight units. As specific examples, Korean Patent Publication Nos. 2001-0012340 and 10-2005-0010760 disclose optical products prepared from polymerizable compositions containing brominated monomers with a high refractive index.

However, the existing halogen-containing compounds are highly toxic and produce a lot of corrosive gases and exhaust gases when they are burned. And, the existing sulfur-containing compounds tend to experience decrease in transitivity with time because of yellowing caused by oxidation. Although a lot of researches are carried out to solve these problems, no satisfactory non-halogen acrylates have been developed as yet and Korea depends entirely on imports.

Further, a high refractive resin composition for a prism layer of a prism sheet needs to be stable against UV, retain a sufficient adhesion to a transparent substrate film, and have a strong surface strength. In addition, it is desired that it exists as liquid at room temperature since processing is difficult if the high refractive composition has an excessively high viscosity.

Accordingly, there is a strong need for development of a novel high refractive organic material that can satisfy the aforesaid conditions and can be produced cost-competitively.

### SUMMARY

The present invention is directed to providing a novel acrylate compound having a high refractive index.

The present invention is also directed to providing a method for preparing the acrylate compound having a high refractive index.

The present invention is also directed to providing a thermosetting resin composition including the acrylate compound.

The present invention is also directed to providing an optical film prepared using the thermosetting resin composition.

The present invention is also directed to providing a display device employing the optical film.

In one general aspect, the present invention provides an acrylate compound represented by Chemical Formula (1) or (2):

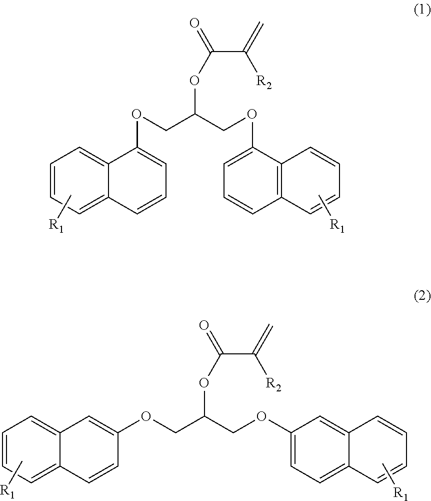

wherein R₁ is selected from a group consisting of H, C₁-C₆ alkyl, —OR′, —NR′, —CN, —NO₂, —CHO, —COR′ and —COOR′ (where R′ is methyl, ethyl or propyl), and R₂ is H or CH₃.

According to an embodiment of the present invention, the acrylate having a high refractive index compound may be 1,3-bis(1-naphthoxy)propan-2-yl acrylate represented by Chemical Formula (3) or 1,3-bis(2-naphthoxy)propan-2-yl acrylate represented by Chemical Formula (4):

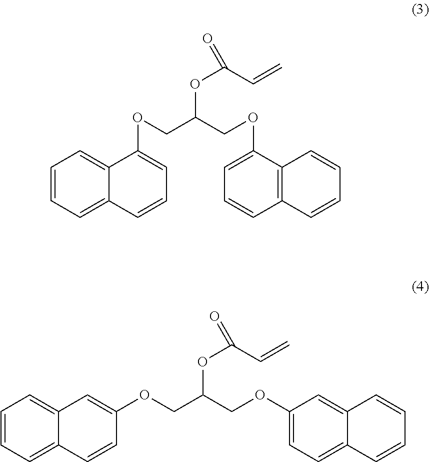

In another general aspect, the present invention provides a method for preparing the acrylate compound represented by Chemical Formula (3) or Chemical Formula (4) from 1,3-bis(1-naphthoxy)-2-propanol represented by Chemical Formula (5) or 1,3-bis(2-naphthoxy)-2-propanol represented by Chemical Formula (6) according to the following scheme:

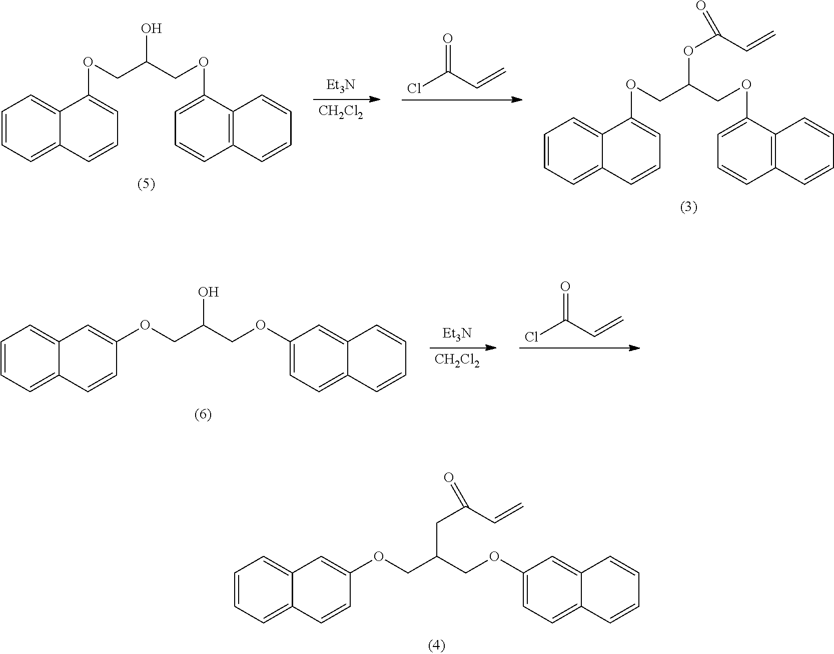

According to an embodiment of the present invention, 1,3-bis(1-naphthoxy)-2-propanol represented by Chemical Formula (5) may be prepared by reacting 1-naphthyl glycidyl ether represented by Chemical Formula (7) with 1-naphthol represented by Chemical Formula (8):

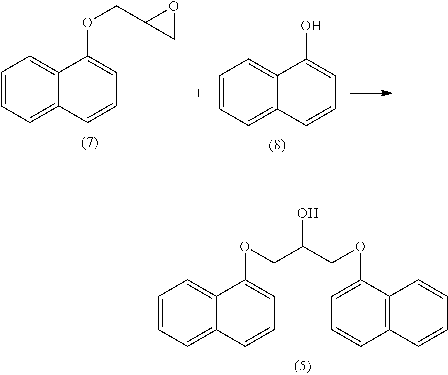

And, 1,3-bis(1-naphthoxy)-2-propanol represented by Chemical Formula (6) may be prepared by reacting 2-naphthyl glycidyl ether represented by Chemical Formula (9) with 2-naphthol represented by Chemical Formula (10):

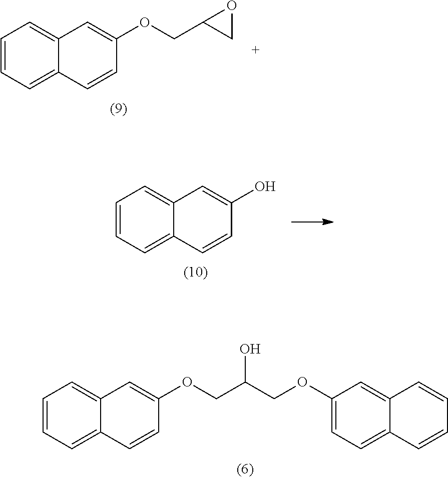

In another general aspect, the present invention provides a photocurable resin composition including: 40 to 60 wt % of an acrylate monomer represented by Chemical Formula (1) or (2); 30 to 50 wt % of a reactive acrylate monomer having one or more functional group(s); and 1 to 5 wt % of a photopolymerization initiator.

According to an embodiment of the present invention, the reactive acrylate monomer having one or more functional group(s) may be one or more compound(s) selected from a group consisting of phenoxy ethyl acrylate, phenoxy diethylene glycol acrylate, phenoxytetraethylene glycol acrylate, phenoxyhexaethylene glycol acrylate, dicyclopentadiene acrylate, 4-hydroxybutyl acrylate, cyclohexanedimethanol monoacrylate, tripropylene glycol diacrylate, polyethylene glycol diacrylate, tris(2-hydroxyethyl)isocyanurate diacrylate, dimethylol tricyclodecane diacrylate, ethylene oxide-added bisphenol A diacrylate, ethylene oxide 3 mol-added trimethylolpropane triacrylate, ethylene oxide 6 mol-added trimethylolpropane triacrylate, pentaerythritol triacrylate, tris(acryloxyethyl) isocyanurate, dipentaerythritol hexaacrylate and caprolactone-modified dipentaerythritol hexaacrylate.

According to another embodiment of the present invention, the photopolymerization initiator may be one or more compound(s) selected from a group consisting of benzophenone, benzophenone derivatives, benzoin, benzoin alkyl ethers, benzyl dimethyl ketals, 1-hydroxycyclohexyl phenyl ketone, diethoxyacetophenone, phosphine oxides, aminoacetophenone, 2-hydroxy-2-methyl-1-phenylpropan-1-one and 2-benzyl-2-dimethylamino-1-(4-morpholinophenyl)-butanone.

In another general aspect, the present invention provides an optical film prepared using a photocurable resin composition comprising the acrylate compound represented by Chemical Formula (1) or (2). The optical film may be, for example, a prism sheet.

In another general aspect, the present invention provides a display device employing the optical film.

Since the novel acrylate compound according to the present invention has a very high refractive index, it is widely applicable to components of display devices such as prism sheet. Further, it is economically and commercially promising since it may be prepared simply by acrylation of aromatic compounds, which are relatively inexpensive and allow recovery of unreacted materials. In addition, it is advantageous in that it is an environment-friendly non-halogen material.

Other features and aspects will be apparent from the following detailed description, the drawings, and the claims.

### BRIEF DESCRIPTION OF THE DRAWINGS

The above and other objects, features and advantages of the present invention will become apparent from the following description of preferred embodiments given in conjunction with the accompanying drawings, in which:

FIG. 1 shows a ¹H NMR spectrum of 1-naphthyl glycidyl ether;

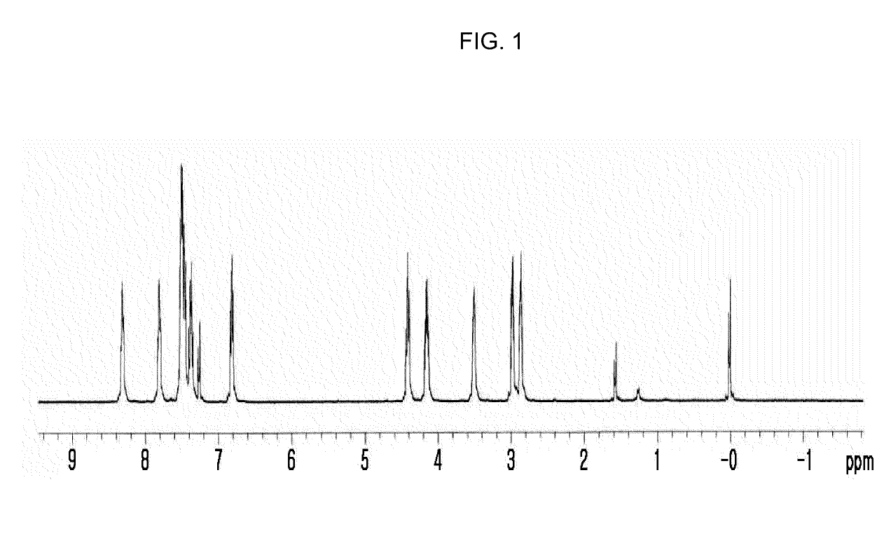

FIG. 2 shows a ¹H NMR spectrum of 2-naphthyl glycidyl ether;

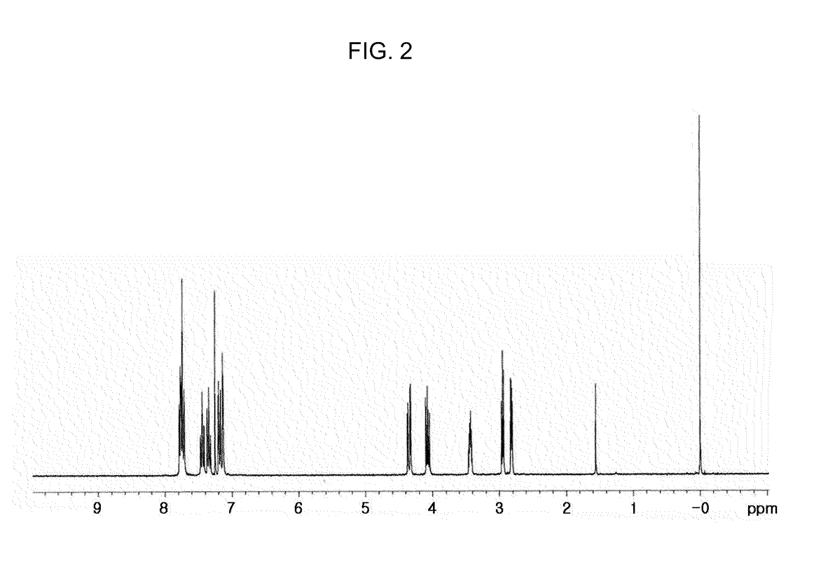

FIG. 3 shows a ¹H NMR spectrum of 1,3-bis(1-naphthoxy)-2-propanol;

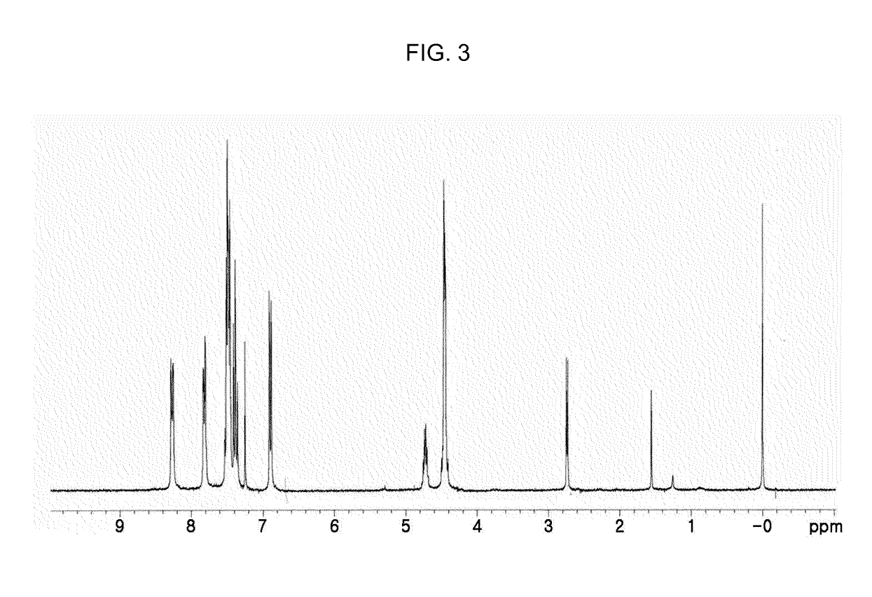

FIG. 4 shows a ¹H NMR spectrum of 1,3-bis(2-naphthoxy)-2-propanol;

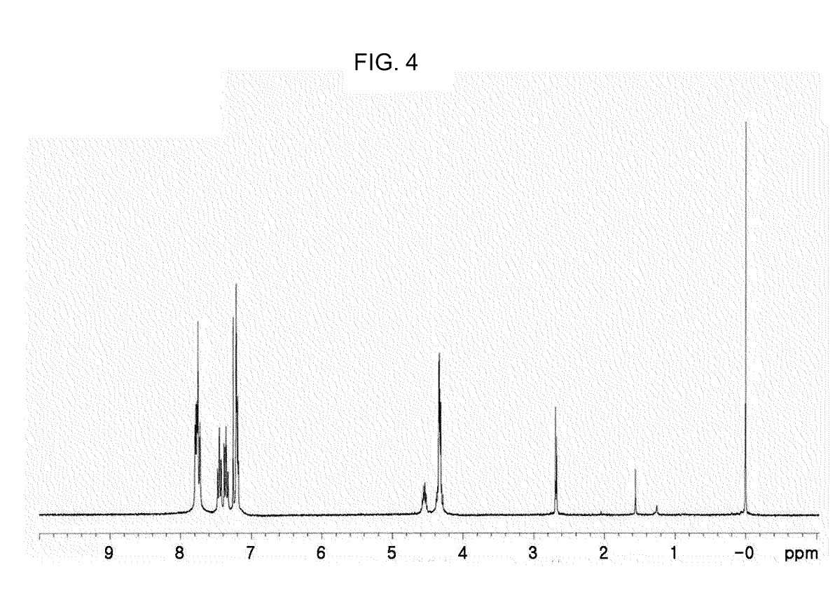

FIG. 5 shows a ¹H NMR spectrum of 1,3-bis(1-naphthoxy)propan-2-yl acrylate;

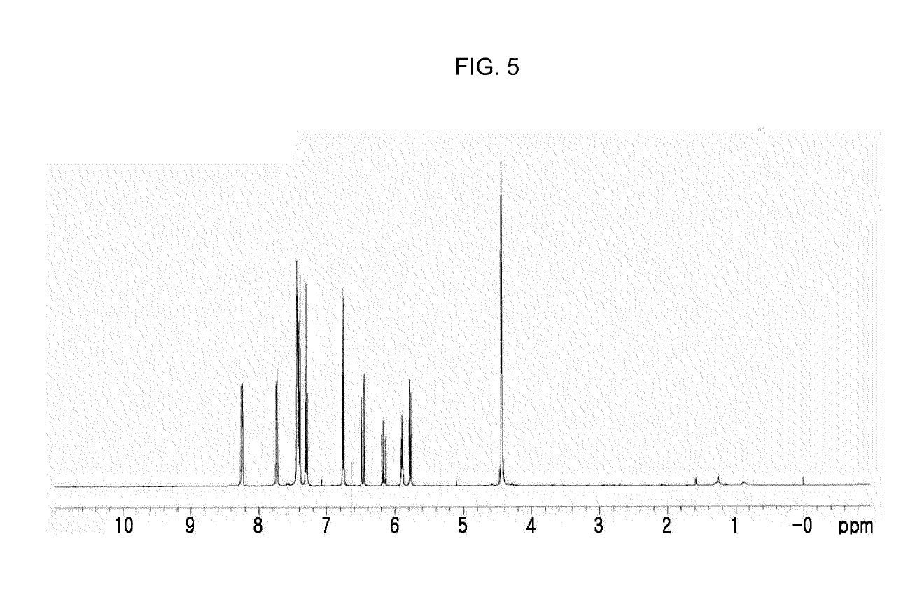

FIG. 6 shows a ¹H NMR spectrum of 1,3-bis(2-naphthoxy)propan-2-yl acrylate;

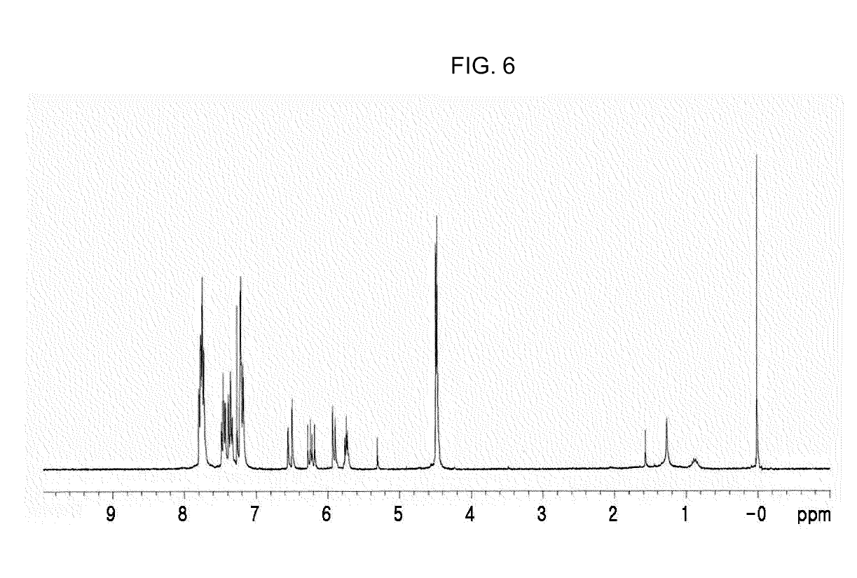

FIG. 7 shows a ¹³C NMR spectrum of 1,3-bis(1-naphthoxy)propan-2-yl acrylate;

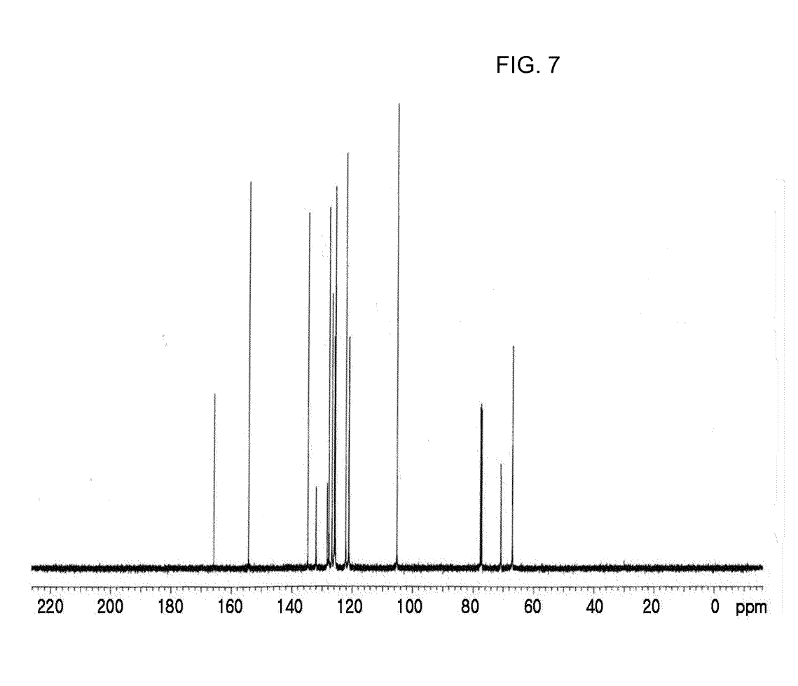

FIG. 8 shows a ¹³C NMR spectrum of 1,3-bis(2-naphthoxy)propan-2-yl acrylate;

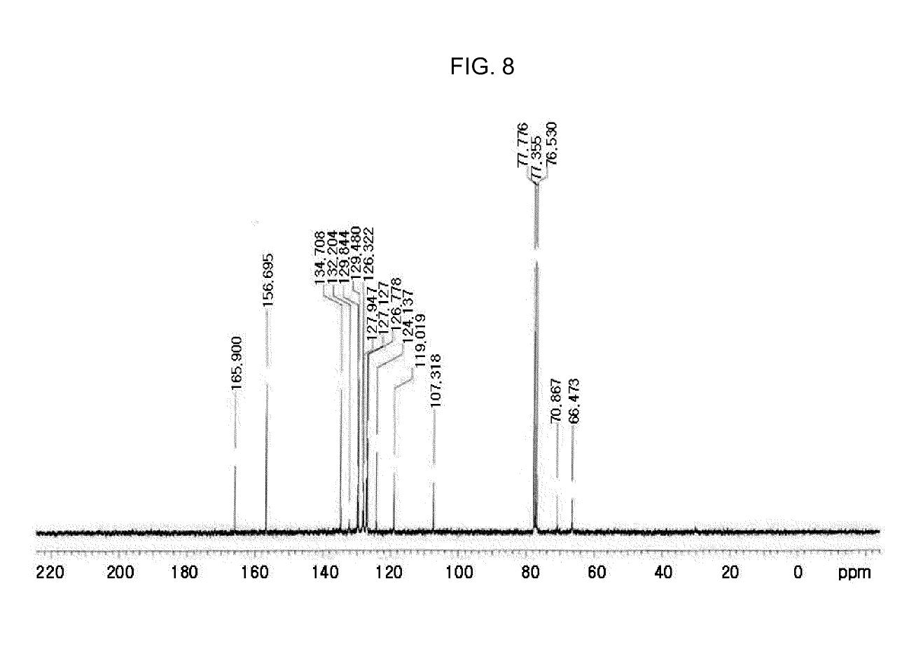

FIG. 9 shows an IR spectrum of 1,3-bis(1-naphthoxy)propan-2-yl acrylate;

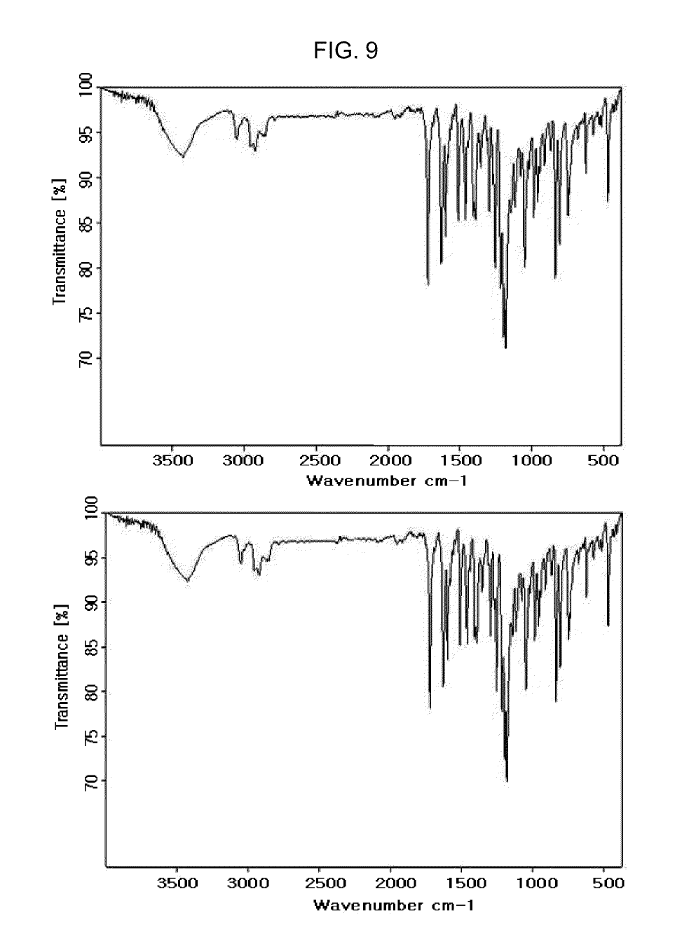

FIG. 10 shows an IR spectrum of 1,3-bis(2-naphthoxy)propan-2-yl acrylate;

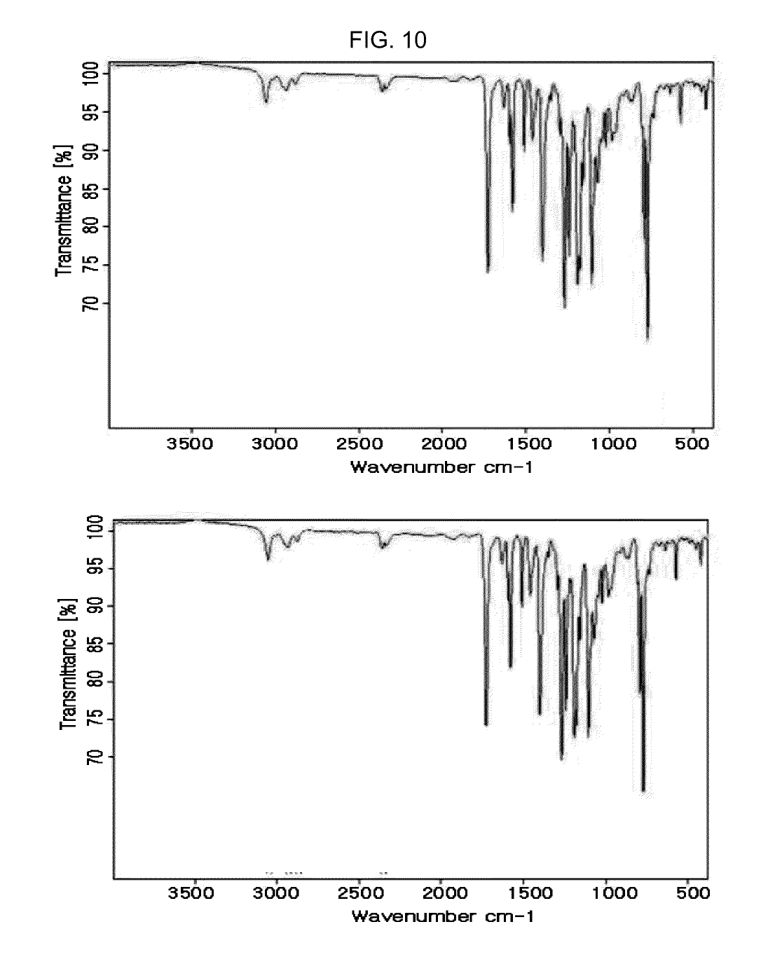

FIG. 11 shows a mass spectrum of 1,3-bis(1-naphthoxy)propan-2-yl acrylate; and

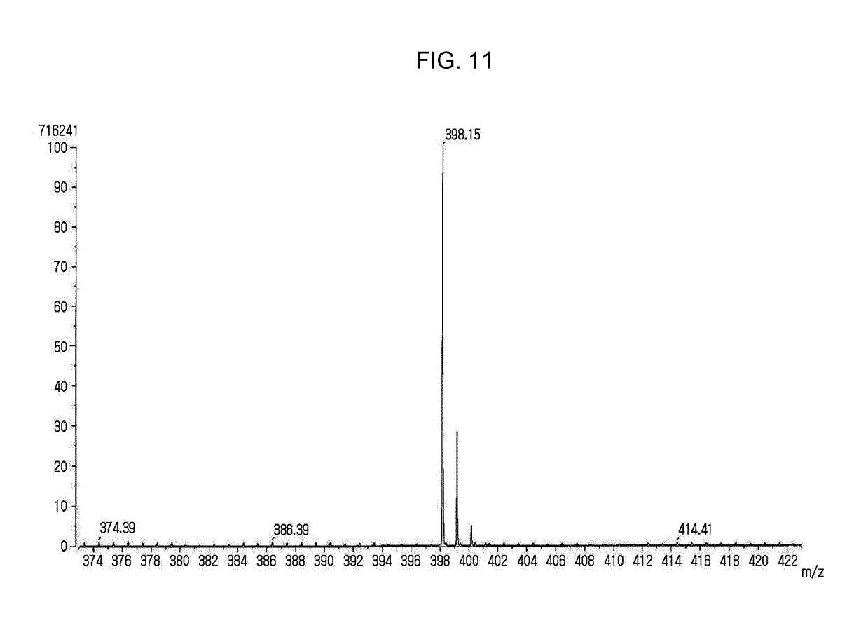

FIG. 12 shows a mass spectrum of 1,3-bis(2-naphthoxy)propan-2-yl acrylate.

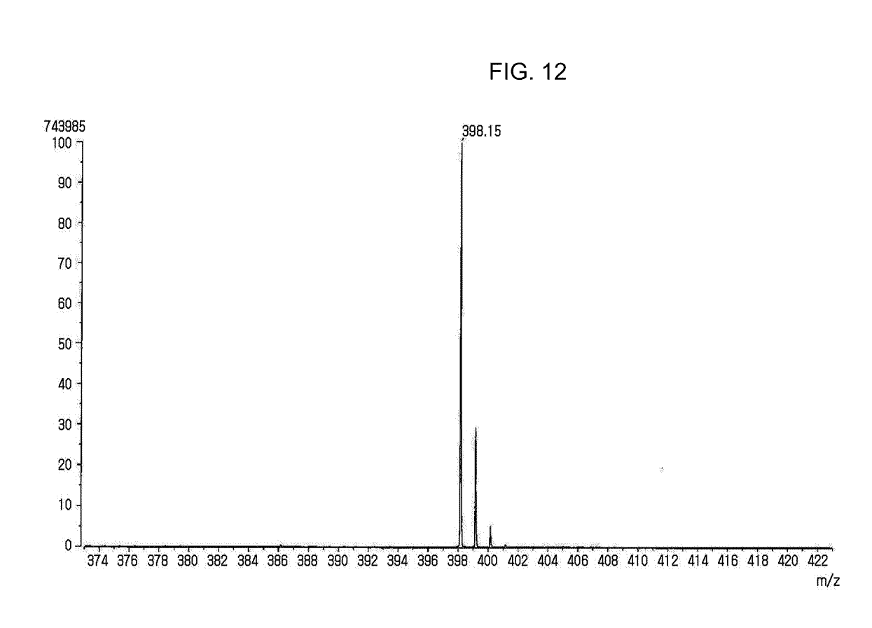

### DETAILED DESCRIPTION OF EMBODIMENTS

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art. The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, exemplary embodiments will be described in detail.

Compounds having aromatic functional groups have been preferred as starting materials since the planar structure of the aromatic compounds, e.g. naphthalene, allow very close intermolecular stacking. Such a dense structure results in increased electron density, which may lead to improvement in glass transition temperature (T_{g}) and refractive index. Based on this fact, the inventors of the present invention have synthesized a glycidyl ether of an aromatic compound and then synthesized a high refractive compound through acrylation.

The present invention provides an acrylate compound represented by Chemical Formula (1) or (2):

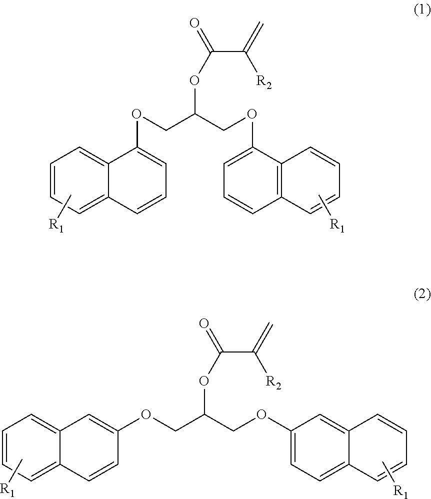

wherein R₁ is selected from a group consisting of H, C₁-C₆ alkyl, —OR′, —NR′, —CN, —NO₂, —CHO, —COR′ and —COOR′ (where R′ is methyl, ethyl or propyl), and R₂ is H or CH₃.

According to an embodiment of the present invention, the acrylate having a high refractive index compound may be 1,3-bis(1-naphthoxy)propan-2-yl acrylate represented by Chemical Formula (3) or 1,3-bis(2-naphthoxy)propan-2-yl acrylate represented by Chemical Formula (4):

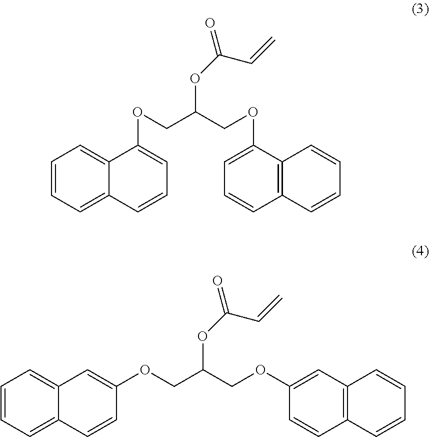

According to an embodiment of the present invention, the acrylate compound represented by Chemical Formula (3) or Chemical Formula (4) may be prepared from 1,3-bis(1-naphthoxy)-2-propanol represented by Chemical Formula (5) or 1,3-bis(2-naphthoxy)-2-propanol represented by Chemical Formula (6) according to the following scheme:

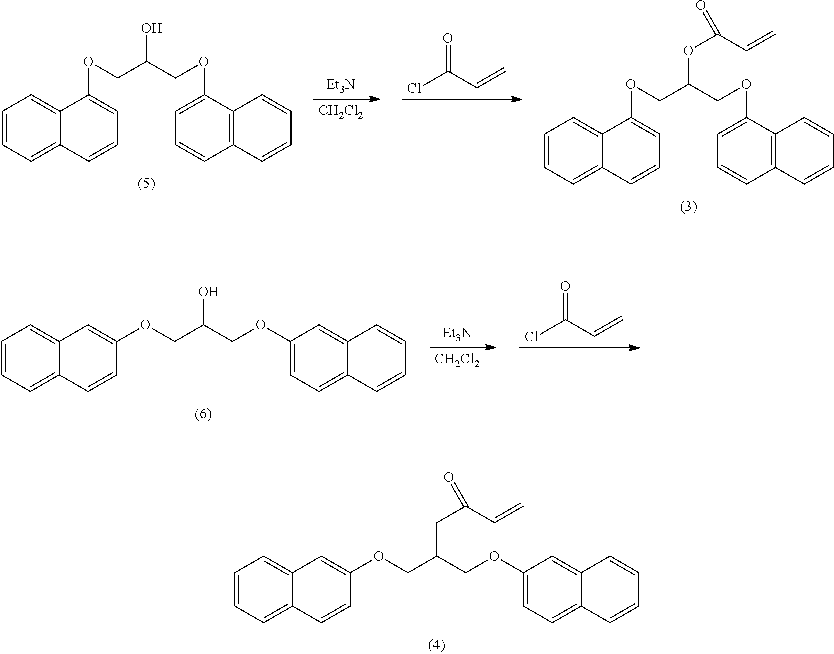

According to an embodiment of the present invention, 1,3-bis(1-naphthoxy)-2-propanol represented by Chemical Formula (5) may be prepared by reacting 1-naphthyl glycidyl ether represented by Chemical Formula (7) with 1-naphthol represented by Chemical Formula (8):

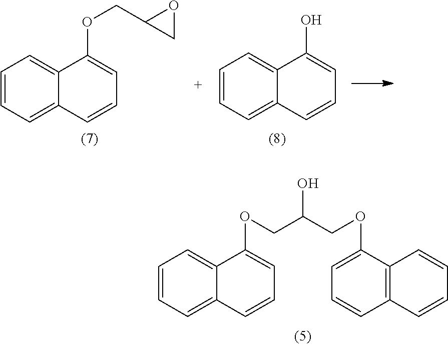

According to another embodiment of the present invention, 1,3-bis(1-naphthoxy)-2-propanol represented by Chemical Formula (6) may be prepared by reacting 2-naphthyl glycidyl ether represented by Chemical Formula (9) with 2-naphthol represented by Chemical Formula (10):

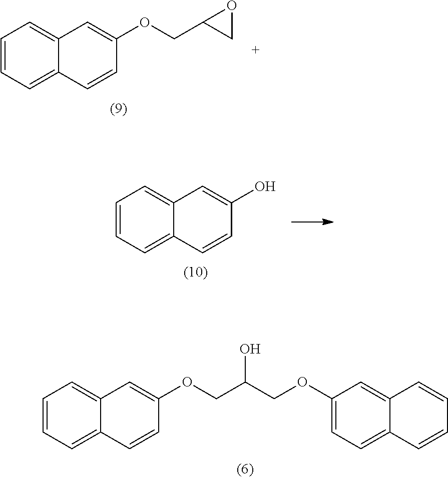

The present invention also provides a photocurable resin composition including: 40 to 60 wt % of an acrylate monomer represented by Chemical Formula (1) or (2); 30 to 50 wt % of a reactive acrylate monomer having one or more functional group(s); and 1 to 5 wt % of a photopolymerization initiator. If the reactive acrylate monomer is used in an amount exceeding 50 wt %, a problem may occur during film casting because of increased viscosity. And, if the photopolymerization initiator is used an amount exceeding 5 wt %, physical properties including refractive index may be unsatisfactory. Hence, the aforesaid range is preferred.

According to an embodiment of the present invention, the reactive acrylate monomer having one or more functional group(s) may be one or more compound(s) selected from a group consisting of phenoxy ethyl acrylate, phenoxy diethylene glycol acrylate, phenoxytetraethylene glycol acrylate, phenoxyhexaethylene glycol acrylate, dicyclopentadiene acrylate, 4-hydroxybutyl acrylate, cyclohexanedimethanol monoacrylate, tripropylene glycol diacrylate, polyethylene glycol diacrylate, tris(2-hydroxyethyl)isocyanurate diacrylate, dimethylol tricyclodecane diacrylate, ethylene oxide-added bisphenol A diacrylate, ethylene oxide 3 mol-added trimethylolpropane triacrylate, ethylene oxide 6 mol-added trimethylolpropane triacrylate, pentaerythritol triacrylate, tris(acryloxyethyl)isocyanurate, dipentaerythritol hexaacrylate and caprolactone-modified dipentaerythritol hexaacrylate.

According to another embodiment of the present invention, the photopolymerization initiator may be one or more compound(s) selected from a group consisting of benzophenone, benzophenone derivatives, benzoin, benzoin alkyl ethers, benzyl dimethyl ketals, 1-hydroxycyclohexyl phenyl ketone, diethoxyacetophenone, phosphine oxides, aminoacetophenone, 2-hydroxy-2-methyl-1-phenylpropan-1-one and 2-benzyl-2-dimethylamino-1-(4-morpholinophenyl)-butanone.

The present invention also provides an optical film prepared using a photocurable resin composition comprising the acrylate compound represented by Chemical Formula (1) or (2). The optical film may be, for example, a prism sheet.

The present invention further provides a display device employing the optical film.

### EXAMPLES

The examples and experiments will now be described. The following examples and experiments are for illustrative purposes only and not intended to limit the scope of this disclosure.

### Example 1-(1)

### Synthesis of 1-naphthyl glycidyl ether using 1-naphthol

1-Naphthol (0.721 g, 5.0 mmol), as a starting material, was stirred with KOH (0.295 g, 5.0 mmol) and methanol (5 mL) in a 50 mL round-bottom flask. When the reagents were completely dissolved in the reaction solvent, the solvent was completely removed through evaporation and freeze-pump-thaw cycling. A dried potassium salt was obtained. After immersing the flask in an oil bath heated to 60 to 90° C., epichlorohydrin (3.91 mL, 10.0 mmol) was added both as a solvent and a reagent. After carrying out reaction at 60° C. for 30 minutes, the reaction solution was washed with brine. A product was extracted from the aqueous solution using a sufficient amount of CH₂Cl₂. After drying with Na₂SO₄, the solvent was removed under reduced pressure. The resulting crude product was purified by silica gel column chromatography (R_{f}=0.20, hexane:ethyl acetate=16:1, v/v). 1-Naphthyl glycidyl ether (0.881 g, 88%) was yielded.

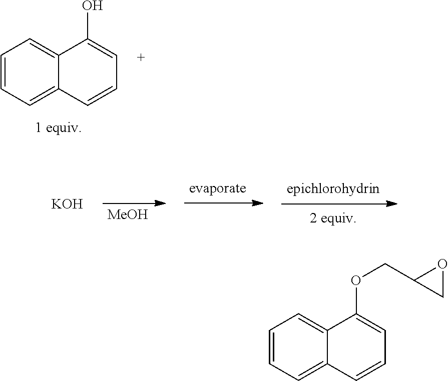

¹H NMR(CHCl₃): δ 8.31 (m, 1H, H at ArH), 7.82 (m, 1H, H at ArH), 7.49 (m, 3H, Hs at ArH), 7.38 (m, 1H, H at ArH), 6.82 (m, 1H, H at ArH), 4.41 (m, 1H, H at C-3), 4.15 (m, 1H, H at C-3), 3.50 (m, 1H, H at C-2), 2.98 (m, 1H, H at C-1), 2.87 (m, 1H, H at C-1).

### Example 1-(2)

### Synthesis of 1,3-bis(1-naphthoxy)-2-propanol using 1-naphthyl glycidyl ether

1-Naphthyl glycidyl ether (1.0 g, 5.0 mmol), as a starting material, was stirred with tetrabutylammonium bromide (0.322 g, 1 mmol) and 1-naphthol (0.865 g, 6 mmol) in a 50 mL round-bottom flask. Then, reaction was carried out at 110° C. for 4 hours and 30 minutes in a toluene solvent (20 mL) under reflux. The resulting crude product was purified by silica gel column chromatography (R_{f}=0.15, hexane:ethyl acetate=9:1, v/v). 1,3-Bis(1-naphthoxy)-2-propanol (1.55 g, 90%) was yielded.

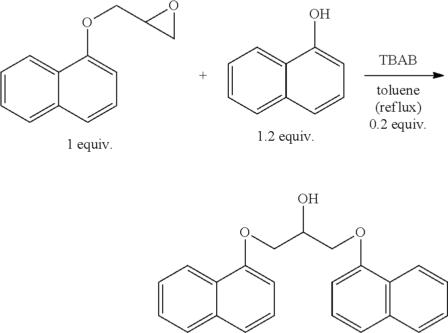

¹H NMR (CDCl₃): δ 8.27 (m, 2H, Hs at ArH), 7.82 (m, 2H, Hs at ArH), 7.49 (m, 6H, Hs at ArH), 7.38 (t, J=7.2 Hz, 2H, Hs at ArH), 6.90 (d, J=7.2 Hz, 2H, Hs at ArH), 4.73 (m, 1H, H at C-2), 4.46 (m, 4H, Hs at C-1), 2.74 (d, J=5.4 Hz, 1H, Hs at OH).

### Example 1-(3)

### Synthesis of 1,3-bis(1-naphthoxy)propan-2-yl acrylate using 1,3-bis(1-naphthoxy)-2-propanol

1,3-Bis(1-naphthoxy)-2-propanol (0.7045 g, 2.0 mmol), as a starting material, was dissolved in a small amount of methylene chloride (10 mL) in a 50 mL round-bottom flask and then stirred after adding triethylamine (0.5817 mL, 4.09 mmol). At 0° C., acryloyl chloride (0.2553 mL, 3.068 mmol) was added dropwise. After carrying out reaction at 0° C. for 20 minutes, followed by washing of the reaction solution with NaHCO₃ aqueous solution, a product was extracted from the aqueous solution using a sufficient amount of methylene chloride.

After drying with MgSO₄, the solvent was removed under reduced pressure. The resulting crude product was purified by silica gel column chromatography (R_{f}=0.25, hexane:ethyl acetate=15:1, v/v). 1,3-Bis(1-naphthoxy)propan-2-ylacrylate (0.6074 g, 71%) was yielded.

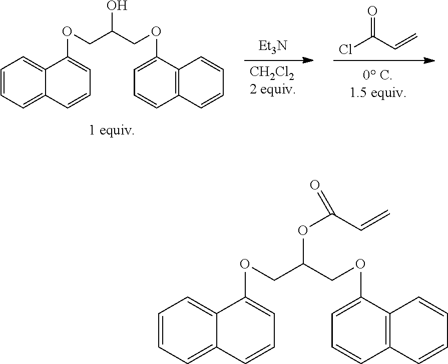

IR (Thin film): 3053.56, 1726.88 cm⁻¹

¹H NMR (CDCl₃): δ 8.25 (m, 2H, Hs at ArH), 7.74 (m, 2H, Hs at ArH), 7.42 (m, 6H, Hs at ArH), 7.30 (m, 2H, Hs at ArH), 6.76 (d, J=7.5 Hz, 2H, Hs at ArH), 6.47 (dd, J=17.1, 1 Hz, 1H, H at C-3′ 6.16 (dd, J=17.4, 10.5 Hz, 1H, H at C-2′), 5.89 (m, 1H, H at 0-2), 5.77 (dd, J=10.5, 1 Hz, 1H, H at C-3′ 4.44 (m, 4H, Hs at C-1).

¹³C NMR (CDCl₃): δ165.79, 154.27, 134.70, 131.99, 128.22, 127.70, 126.73, 125.96, 125.75, 125.64, 122.10, 121.15, 105.18, 70.74, 66.97

High Res. Mass data: C₂₆H₂₂O₄, Calc. 398.1518. Found 398.1516

### Example 2-(1)

### Synthesis of 2-naphthyl glycidyl ether using 2-naphthol

2-Naphthol (3.67 g, 25.0 mmol), as a starting material, was stirred with KOH (1.48 g, 25.0 mmol) and methanol (30 mL) in a 250 mL round-bottom flask. When the reagents were completely dissolved in the reaction solvent, the solvent was completely removed through evaporation and freeze-pump-thaw cycling. A dried potassium salt was obtained. After immersing the flask in an oil bath heated to 60 to 90° C., epichlorohydrin (19.56 mL, 250 mmol) was added both as a solvent and a reagent. After carrying out reaction at 60° C. for 30 minutes, the reaction solution was washed with brine. A product was extracted from the aqueous solution using a sufficient amount of CH₂Cl₂. After drying with Na₂SO₄, the solvent was removed under reduced pressure. The resulting crude product was purified by silica gel column chromatography (R_{f}=0.20, hexane:ethyl acetate=16:1, v/v). 2-Naphthyl glycidyl ether (4.615 g, 92%) was yielded.

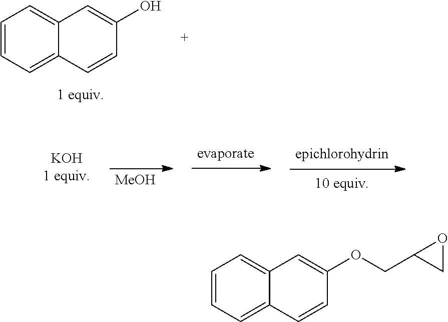

¹H NMR (CDCl₃): δ 7.75 (m, 3H, Hs at ArH), 7.44 (m, 1H at ArH), 7.35 (m, 1H, ArH), 7.17 (m, 2H, Hs at ArH), 4.35 (dd, J=11.1, 3 Hz, 1H, H at C-3), 4.07 (dd, J=11.3, 6 Hz, 1H, H at C-3), 3.44 (m, 1H, H at C-2), 2.95 (t, J=4.5 Hz, 1H, Hs at C-1), 2.82 (q, J=2.4 Hz, 1H, H at C-1).

### Example 2-(2)

### Synthesis of 1,3-bis(2-naphthoxy)-2-propanol using 2-naphthyl glycidyl ether

2-Naphthyl glycidyl ether (1.0 g, 5.0 mmol), as a starting material, was stirred with tetrabutylammonium bromide (0.081 g, 0.25 mmol) and 2-naphthol (0.441 g, 3 mmol). Then, reaction was carried out at 110° C. for 2 hours and 30 minutes in a toluene solvent (20 mL) under reflux. The resulting crude product was purified by silica gel column chromatography (R_{f}=0.2, hexane:ethyl acetate=5:1, v/v). 1,3-Bis(2-naphthoxy)-2-propanol (0.7579 g, 88%) was yielded.

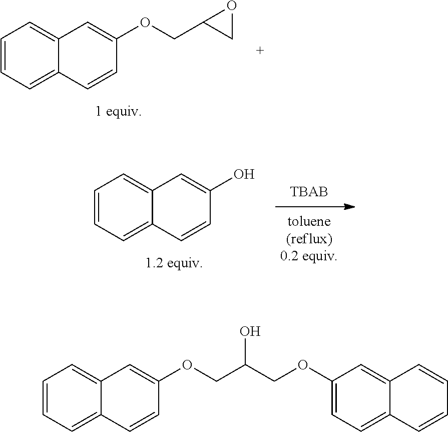

¹H NMR (CDCl₃): δ 7.76 (m, 6H, Hs at ArH), 7.45 (td, J=7.2, 1.5 Hz, 2H, Hs at ArH), 7.35 (m, 2H, Hs at ArH), 7.20 (m, 4H, Hs at ArH), 4.54 (m, 1H, Hs at C-2), 4.33 (m, 4H, Hs at C-1), 2.68 (d, J=4.8 1H, Hs at OH).

### Example 2-(3)

### Synthesis of 1,3-bis(2-naphthoxy)propan-2-yl acrylate using 1,3-bis(2-naphthoxy)-2-propanol

1,3-Bis(2-naphthoxy)-2-propanol (0.5166 g, 1.5 mmol), as a starting material, was dissolved in a small amount of methylene chloride (7.5 mL) in a 50 mL round-bottom flask and then stirred after adding triethylamine (0.4243 mL, 3 mmol). At 0° C., acryloyl chloride (0.2553 mL, 3.068 mmol) was added dropwise. After carrying out reaction at 0° C. for 30 minutes, followed by washing of the reaction solution with NaHCO₃ aqueous solution, a product was extracted from the aqueous solution using a sufficient amount of methylene chloride. After drying with MgSO₄, the solvent was removed under reduced pressure. The resulting crude product was purified by silica gel column chromatography (R_{f}=0.3, hexane:ethyl acetate=10:1, v/v). 1,3-Bis(2-naphthoxy)propan-2-yl acrylate (0.47 g, 75%) was yielded.

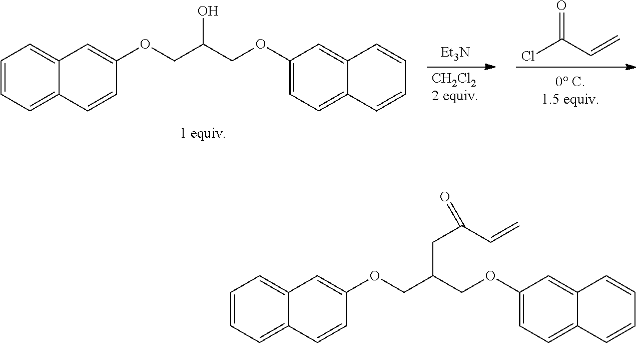

IR (KBR pellet): 3423.75, 1722.20 cm⁻¹

¹H NMR (CDCl₃): δ 7.77 (m, 5H, H at ArH), 7.45 (m, 2H, Hs at ArH), 7.36 (m, 2H, Hs at ArH), 7.20 (m, 4H, Hs at ArH), 6.53 (dd, J=17.1, 1.5 Hz, 1H, H at C-3′), 6.23 (dd, J=17.4, 10.2 Hz, 1H, H at C-2′), 5.92 (dd, J=10.2, 1.5 Hz, 1H at C-3′), 5.75 (m, 1H, H at C-1), 4.49 (d, J=5.4 Hz, 4H, Hs at C-2).

¹³C NMR (CDCl₃): δ165.90, 156.64, 134.71, 132.20, 129.84, 129.48, 128.32, 127.95, 127.13, 126.78, 124.19, 119.02, 107.32, 70.87, 66.47.

High Res. Mass data: C₂₆H₂₂O₄, Calc. 398.1518. Found 398.1519.

### Test Example 1

### Measurement of Refractive Index

Refractive indices of 1,3-bis(1-naphthoxy)propan-2-yl acrylate prepared in Example 1 and 1,3-bis(2-naphthoxy)propan-2-yl acrylate prepared in Example 2 were measured using an Abbe refractometer NAR-1T Solid under 20° C. neat condition. The measurement values were very high when compared with existing acrylates. Accordingly, a significant improvement in brightness is expected when the acrylates are used, for example, for an optical film.

TABLE 1

| | | Refractive |
|---|---|---|
| | Chemical formula | index |
| Example 1: 1,3-bis(1-naphthoxy) propan-2-yl acrylate | 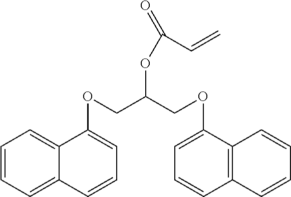 | 1.6259 |
| Example 2: 1,3-bis(2-naphthoxy) propan-2-yl acrylate | 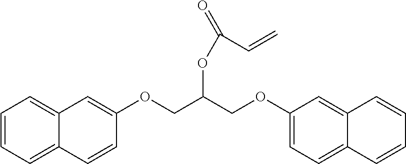 | 1.6208 |

### Test Example 2

### Measurement of Melting Point

Melting points of the acrylates prepared in Example 1 and Example 2 were measured. 1,3-Bis(1-naphthoxy)propan-2-yl acrylate was a viscous solid material, and the melting point of 1,3-bis(2-naphthoxy)propan-2-yl acrylate was measured as 122 to 123° C.

### Example 3

### Preparation of Photocurable Resin Composition

Photocurable resin compositions were prepared mixing 40 to 60 wt % of the acrylate monomer synthesized in Example 1 or Example 2 with 30 to 50 wt % of a reactive acrylate monomer having one or more functional group(s) and 1 to 5 wt % of a photopolymerization initiator. Specifically, 50 wt % of 1,3-bis(1-naphthoxy)propan-2-yl or 1,3-bis(2-naphthoxy)propan-2-yl acrylate, 45 wt % of phenoxyhexaethylene glycol acrylate as the reactive acrylate monomer, and 5 wt % of 2-hydroxy-2-methyl-1-phenylpropan-1-one as the photopolymerization initiator were used.

### Example 4

### Preparation of Optical Film

After applying the photocurable resin composition prepared in Example 3 on a coating surface of a transparent substrate film (PET film), the coated composition was photocured by radiating UV along the transparent substrate film. Then, the cured coating layer was separated from the transparent substrate film. As a result, a prism optical film with a prism layer formed thereon was prepared.

While the present invention has been described with respect to the specific embodiments, it will be apparent to those skilled in the art that various changes and modifications may be made without departing from the spirit and scope of the invention as defined in the following claims.

What is claimed is:

1. An acrylate compound represented by Chemical Formula (1): 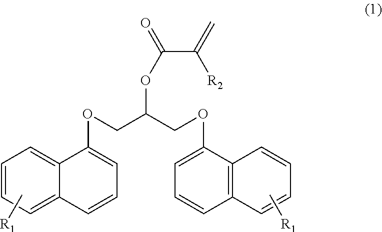
wherein R₁ is selected from a group consisting of H, C₁-C₆ alkyl, —OR′, —NR′, —CN, —NO₂, —CHO, —COR′ and —COOR′ (where R′ is methyl, ethyl or propyl), and R₂ is H or CH₃.

2. The acrylate compound according to claim 1, which is represented by Chemical Formula (3):

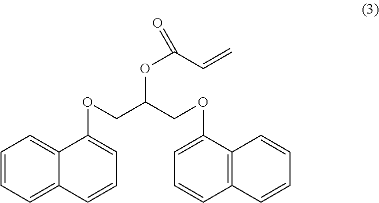

3. An acrylate compound represented by Chemical Formula (2): 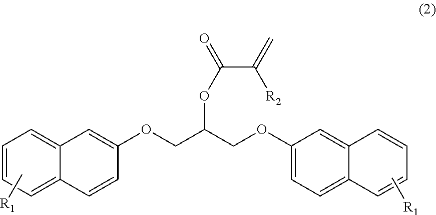
wherein R₁ is selected from a group consisting of H, C₁-C₆ alkyl, —OR′, —NR′, —CN, —NO₂, —CHO, —COR′ and —COOR′ (where R′ is methyl, ethyl or propyl), and R₂ is H or CH₃.

4. The acrylate compound according to claim 3, which is represented by Chemical Formula (4):

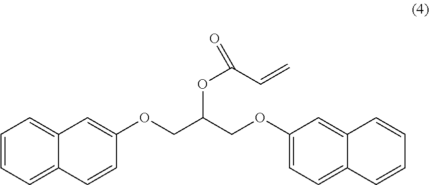

5. A method for preparing the acrylate compound represented by Chemical Formula (3) according to claim 1 from 1,3-bis(1-naphthoxy)-2-propanol represented by Chemical Formula (5) according to the following scheme:

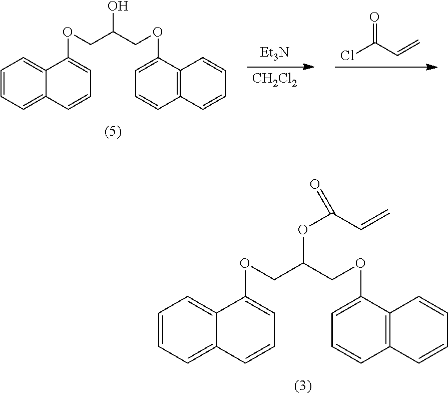

6. The method for preparing the acrylate compound according to claim 5, wherein 1,3-bis(1-naphthoxy)-2-propanol represented by Chemical Formula (5) is prepared by reacting 1-naphthyl glycidyl ether represented by Chemical Formula (7) with 1-naphthol represented by Chemical Formula (8):

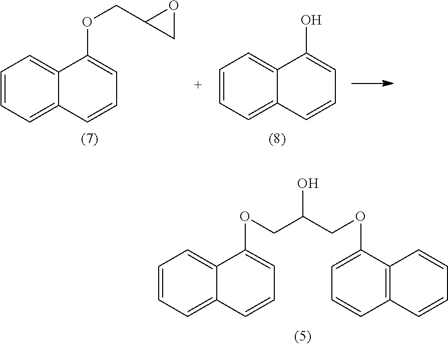

7. A method for preparing the acrylate compound represented by Chemical Formula (4) according to claim 4 from 1,3-bis(2-naphthoxy)-2-propanol represented by Chemical Formula (6) according to the following scheme:

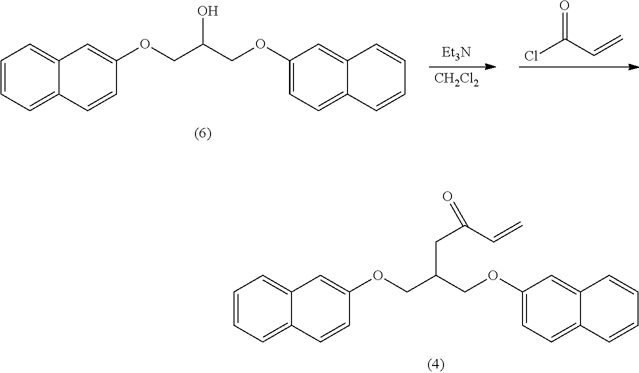

8. The method for preparing the acrylate compound according to claim 7, wherein 1,3-bis(2-naphthoxy)-2-propanol represented by Chemical Formula (6) is prepared by reacting 2-naphthyl glycidyl ether represented by Chemical Formula (9) with 2-naphthol represented by Chemical Formula (10):

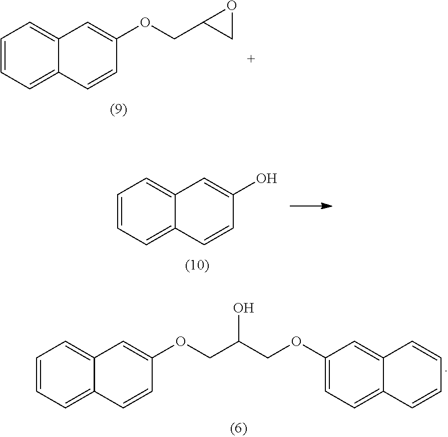

9. A photocurable resin composition comprising:
40 to 60 wt % of an acrylate monomer according to claims 1;
30 to 50 wt % of a reactive acrylate monomer having one or more functional group(s); and
1 to 5 wt % of a photopolymerization initiator.

10. The photocurable resin composition according to claim 9, wherein the reactive acrylate monomer having one or more functional group(s) is one or more compound(s) selected from a group consisting of phenoxy ethyl acrylate, phenoxy diethylene glycol acrylate, phenoxytetraethylene glycol acrylate, phenoxyhexaethylene glycol acrylate, dicyclopentadiene acrylate, 4-hydroxybutyl acrylate, cyclohexanedimethanol monoacrylate, tripropylene glycol diacrylate, polyethylene glycol diacrylate, tris(2-hydroxyethyl)isocyanurate diacrylate, dimethylol tricyclodecane diacrylate, ethylene oxide-added bisphenol A diacrylate, ethylene oxide 3 mol-added trimethylolpropane triacrylate, ethylene oxide 6 mol-added trimethylolpropane triacrylate, pentaerythritol triacrylate, tris(acryloxyethyl)isocyanurate, dipentaerythritol hexaacrylate and caprolactone-modified dipentaerythritol hexaacrylate.

11. The photocurable resin composition according to claim 9, wherein the photopolymerization initiator is one or more compound(s) selected from a group consisting of benzophenone, benzophenone derivatives, benzoin, benzoin alkyl ethers, benzyl dimethyl ketals, 1-hydroxycyclohexyl phenyl ketone, diethoxyacetophenone, phosphine oxides, aminoacetophenone, 2-hydroxy-2-methyl-1-phenylpropan-1-one and 2-benzyl-2-dimethylamide-1-(4-morpholinophenyl)-butanone.

12. An optical film prepared using a photocurable resin composition comprising the acrylate compound according to claim 1.

13. The optical film according to claim 12, which is a prism sheet.

14. A display device employing the optical film according to claim 12.
